(19) United States
(12) Patent Application Publication
(10) Pub. No.: US 2008/0312682 A1
(43) Pub. Date: Dec. 18, 2008

(54) Pressure Indicating Device

(76) Inventors: Iden Shams, London (GB); Anthony Arthur Wills, London (GB); Brian John Watson, London (GB)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 12/067,602

(22) PCT Filed: Sep. 25, 2006
(86) PCT No.: PCT/GB2006/003561

§ 371 Date: Aug. 11, 2008

(30) Foreign Application Priority Data
Sep. 23, 2005 (GB) 0519472.5

(51) Int. Cl. A61B 17/132

(52) U.S. Cl. 606/203

(57) ABSTRACT

A device for indicating the pressure applied to an object by a strap (21). The device is adapted to receive an end of the strap and to allow the strap end to be pulled through the device against the action of a deformable spring means (22), thereby progressively increasing the pressure applied to the object. The spring means progressively deflects in proportion to that applied pressure. A linking means (24) translates the deflection of the spring means to the movement of a pointer (23) which thus provides an indication of the pressure being applied by the strap against some reference markings on the strap (25).

### FIELD OF THE INVENTION

The invention relates to devices for indicating pressure applied by a strap.

There are numerous situations where it would be useful to have an indication that a certain pressure is being exerted by a strap. One is when using a medical tourniquet. Another is when a bundle of cylindrical objects such as pipes is to be lashed together. A third is when the many buckled straps which tighten the tarpaulin sides of a so called curtain sider lorry are to be each tautened to substantially the same pressure on the tarpaulin which cumulatively they hold down.

The present invention is applicable to all three of these situations and more, but will be described primarily with reference to a medical tourniquet.

### Review of Art Known to the Applicant

Medical tourniquets are used to assist in taking blood samples from patients by being wrapped and tightened around a patient's arm. This action restricts blood flow through the relevant vein, making it distend so that it is easier for a nurse or doctor to insert a needle in the vein. The pressure required differs between patients, in particular between children and adults. The force (i.e. the amount of tension in the strap) can lead to discomfort on the part of the patient if too much force is applied and may cause bruising.

Alternatively if insufficient pressure is applied, the vein does not distend and intravenous injection becomes difficult or even impossible.

Known tourniquet devices are often expensive, or are not easy to handle, or both. Woven tapes come into the first category. Simple rubber bands or surgical gloves would fit the second. In essence, assessment of the correct pressure in any given instance must be made by a trained professional using skill learned over a period of time—and with no indication of the pressure actually being applied by the tourniquet to the limb.

Tourniquets are of course also used to apply pressure to restrict or stop blood flow where an artery has been severed. In such cases of emergency there is an absolute need to apply enough pressure to restrict or stop blood flow. A tourniquet which provided a positive visual indication that the appropriate pressure had been applied could safely be used by relatively untrained and inexperienced people.

There is an ancillary problem with tourniquets, in that they can all too easily be used on a number of different patients without being sterilised. This can lead to the transmission of infection. There is therefore an additional need for an easier-to-use tourniquet, which is disposable—ideally one, which is positively rendered useless after one use, only, so that the user is forced to dispose of it and the risk of cross-contamination is ended.

In summary, it would be useful to have a simple device that could provide an indication that the appropriate pressure is being applied by a strap—for example, a tourniquet being used on a patient's limb—and in the case under discussion, preferably a disposable device; and preferably a tourniquet which is a one-use-only tourniquet.

### SUMMARY OF THE INVENTION

In its broadest aspect the invention provides a device for indicating the pressure applied to an object by a strap attached thereto as the strap, in use, is tensioned; the device being adapted to receive an end of the strap and to allow the strap end to be pulled through the device against the action of a deformable spring means with which, in use, the strap end co-operates so that as the strap end is pulled through the device, thereby progressively increasing the pressure applied to the object, the spring means progressively deflects in proportion to that applied pressure; and with means linking the deflection of the spring means to the movement of a pointer which thus provides an indication of the pressure being applied by the strap.

Preferably the spring means is resiliently deformable, thereby enabling the device to be reused with the same or another intact strap in the case where one-use-only sterility is not important, e.g. the pipe-lashing or container lorry examples briefly outlined above.

Advantageously the pointer is orientated to move progressively over the width dimension of the strap as the spring deflection increases. Whilst it is not an essential feature the invention in its broadest aspect, it is clearly visually advantageous, because the width extremities of the moving strap form a natural and literal band of extreme linear visual datum references between which it is easy for a user to gauge the extent of pointer movement from the starting position.

In any embodiment in the invention, the device may with advantage incorporate a non-return gate which, as the strap end is pulled through the device, prevents the strap from loosening its pressure on the object. In this specification we use the term “non-return gate” to define any means that prevents the strap from so loosening its pressure. Such mechanisms are known in themselves—and are frequently referred to as a “one-way ratchet” mechanism—and may be incorporated into the strap to interact with co-operating teeth on the device. But it is also within the scope of the invention to use non-return gates of known kinds which can be incorporated wholly within the device, to grip the strap without any modification of a conventional strap's surface; and which intended skilled addressees of this specification will select without the need for inventive thought.

The spring means itself may be a literal spring, for example a coiled compression spring or tension spring. In the preferred of two embodiments to be described in this specification, the spring is a torsion spring, but it could alternatively consist of a spring means in the form of a resilient elastic band; or such a band might be used to augment the natural action of a literal spring of any of these kinds.

Where the device does incorporate a non-return gate of whatever appropriate kind, it is preferably of the kind which cannot subsequently be released other than by breaking the strap. This promotes the one-use-only philosophy to best effect.

In use, whilst this device can be supplied for retrofitting to any appropriately sized strap of compatible material properties, it is advantageously supplied with a strap particularly adapted to co-operate with it, in which a reference datum is marked on that region of the strap which, in use, will pass through the device and be visible by a user when the strap movement ceases, i.e. when maximum pressure applied has been achieved. An advantage of such a datum—which can be a line, preferably curved, or a series of dots or other appropriate indicating datum—lies in its ability (when the device is appropriately calibrated) to interact visually with the pointer to show whether or not a predetermined pressure has been reached or exceeded. Although in the preferred embodiment the pointer literally intersects the line when this state is achieved, it is enough for the line or other datum to act as just a visual datum, so that if the pointer approaches it, or exceeds it in its travel, then the same effect—e.g. in the hands of a skilled operator—is achieved.

Ideally the strap is so designed that breaking it renders the strap unable to be reused, so the one-use-only philosophy reaches its zenith. This is especially important in the case of medical tourniquets for all the reasons canvassed previously.

In practical embodiments the strap is preferably made from a substantially inextensible material so minimising any risk of the pointer indication being inaccurate. In any other construction, there is a risk of this if, for instance, an exceptionally strong pull on an inherently inextensible strap were to largely elongate the strap and only minimally move it forward.

### BRIEF DESCRIPTION OF THE DRAWINGS

Presently preferred embodiments of the invention will now be described, by way of example only, with reference to the accompanying drawings in which:

FIGS. 1 though 5 are respectively perspective, plan, plan again, plan again, and cross-sectional side views of a first embodiment;

FIGS. 6 though 9 are a similarly respective perspective view, side-section, end-section, and plan views of the second and better embodiment.

In the drawings, FIG. 2 shows the device in plan prior to tension being applied to its strap.

FIG. 3 again shows the device when correct pressure has been applied to an object. FIG. 4 is similar to FIG. 3 but showing the device when the correct pressure has been applied to an object with a different circumference which the device and strap are encircling in the manner of a tourniquet.

FIG. 7 is a cross-section along the line AA of FIG. 9. FIG. 8 is a cross section along the line BB of FIG. 9.

### DESCRIPTION OF THE PREFERRED EMBODIMENTS

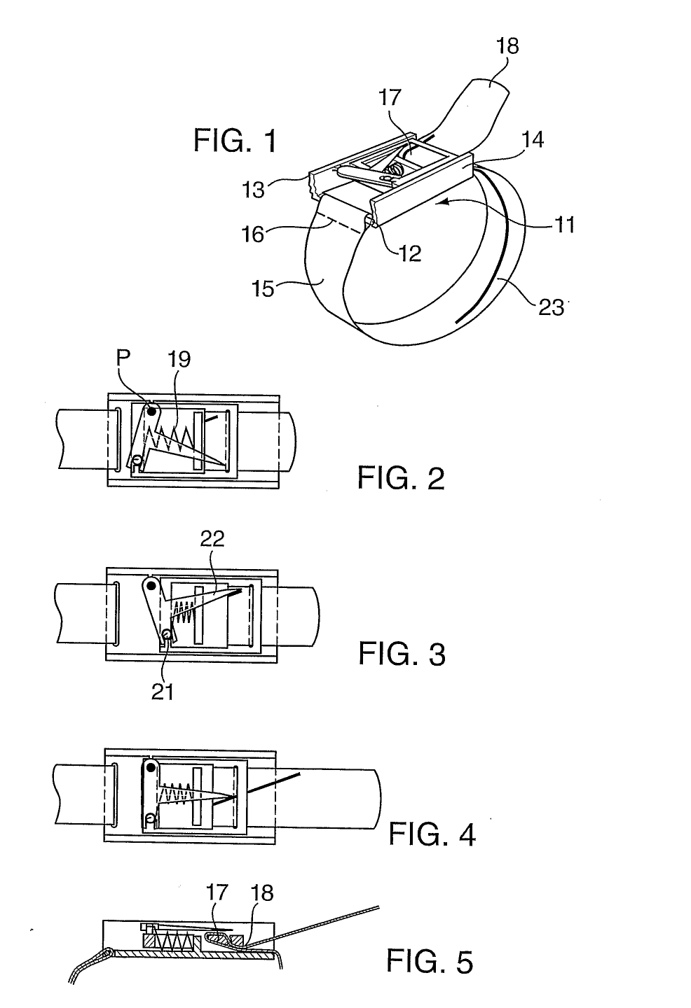

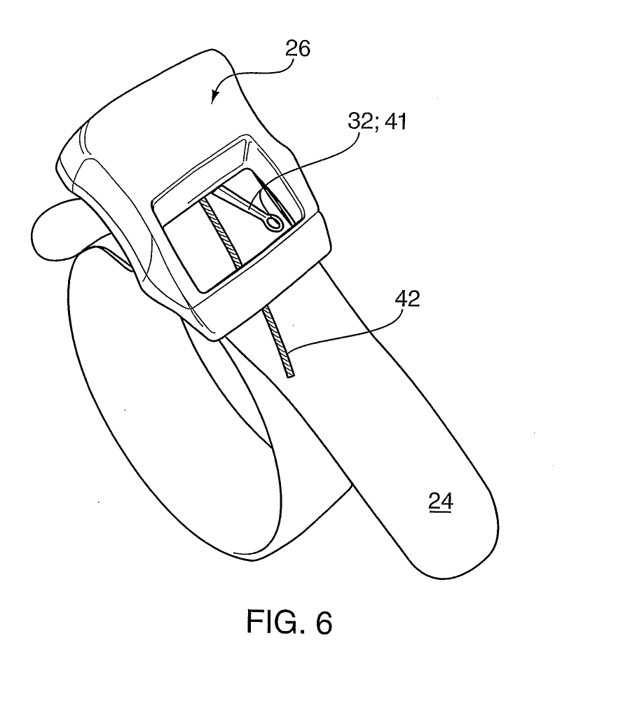

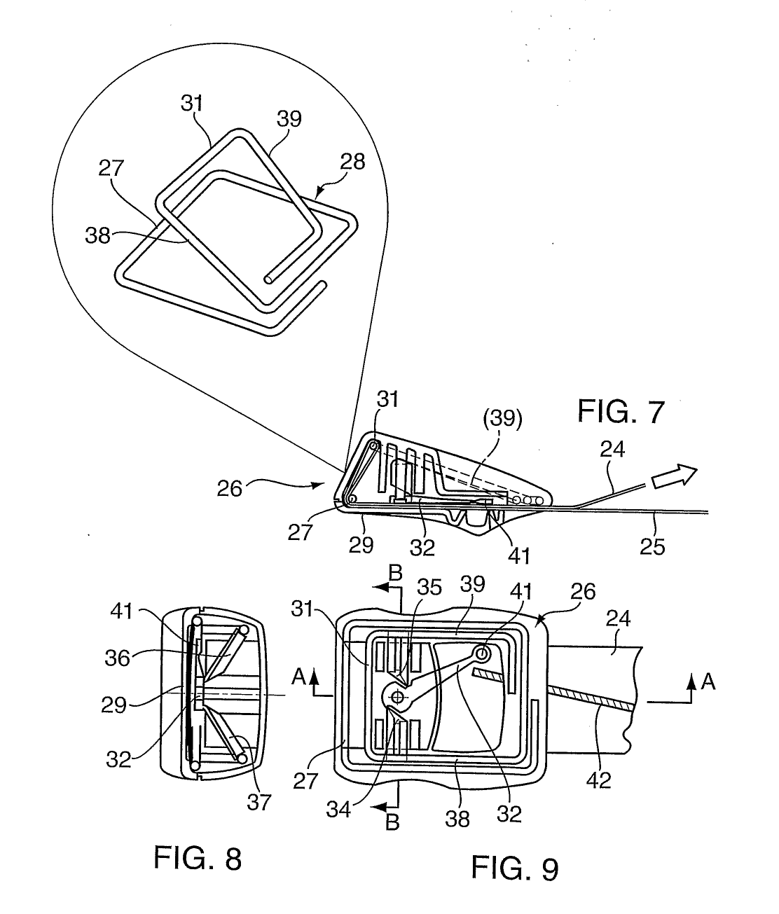

In FIG. 1 a device in the form of a tourniquet buckle is combined with a substantially flat tape strap to form a medical tourniquet. The device is built around a moulded plastics housing 11 consisting essentially of a substantially flat base 12 from each opposite longitudinal edge of which there rises a side wall 13, 14. The device 14 could equally well be used on a linear strap between points and is not restricted just to the looped strap illustrated.

The strap 15 itself is fastened to one end of the device by passing it through a slot, extending substantially the whole of the width of the device between the sidewalls 13 and 14, and then fastening it to itself using a line of ultrasonic spot welds 16 whose purpose will be explained later. The tourniquet loop is formed by passing the other end of the strap through the end of the device opposite to the end around which the spot welds 16 are fastened, through an adjustable connection 17, and out to form a strap end 18 which can be pulled by a user progressively through the device housing 11 and hence progressively tighten the loop around an object to which pressure is to be applied.

The adjustable connection 17 incorporates a non-return gate 18 which can be of known kind, for example the one-way ratchet kind previously mentioned. This ensures that the strap 15 can be pulled by its end 18 through the housing 11 in one direction only. Hence the loop of the tourniquet can only be made smaller not larger.

The adjustable connection 17 is capable of moving to a limited extent, along the length of the housing 11, but is initially held in place by a coiled compression spring 19. Applying tension to the strap 15 by pulling on its end 18 will cause the spring to be either extended or compressed, depending on whether a coiled tension spring or coiled compression spring is fitted. In this embodiment the spring is compressed, allowing the adjustable connection 17 to move in direct proportion to the pressure being applied to the object which the tourniquet surrounds in use.

The adjustable connection 17 is attached via a linkage 21, to a pointer 22. The linkage takes the form of a pin, fixed to one end of the spring opposite that which is attached to the adjustable connection 17, and engaging a slot as illustrated in the pointer base. This linkage converts the longitudinal deflection of the spring, as the strap is pulled, into a transverse movement of the end of the pointer 22 about a pivot “P”. The pointer-end movement is transverse in that it moves across the width dimension of the strap end region as the strap passes through the housing.

A reference datum in the form of a curved line 23 is printed or otherwise marked on the strap. The line curves progressively across the width of the strap as illustrated. The device is calibrated so that a specific tension applied by the strap can be matched to a specific pressure being applied to the object that the tourniquet is place around in such a way that when the pointer coincides with the line as the strap is pulled through the housing, an appropriate predetermined pressure has been reached.

In the particular embodiment illustrated with reference to FIGS. 1 though 5, the tourniquet is deliberately a one-use-only embodiment. To release the tourniquet, a user must pull sharply on the region of the strap adjacent to the spot welds 16. This will break the spot welds and allow the strap to come loose. The non-return gate 18 prevents the user from removing the tourniquet by loosening it at that end. But once the line of spot welds 16 has been broken, the strap cannot be reused.

In the second, embodiment of FIGS. 7 though 9 the strap 25 enters the housing 26 and is threaded around a bar 27 which extends across the width of the housing and is the bottom bar of a torsion spring 28 held inside the base half 29 of the two-half moulded housing 26. The strap is then looped around the top bar 31 of the torsion spring 28 whose opposite legs 38 and 39 extend parallel to one another from the floor of the half-housing 29 internally. The general form of the spring 28 is shown in the scrap view leading off FIG. 7 and it will be appreciated, that as the top bar 31 of the spring is pulled downwards, that pull is exerted against the action of the torsion elements.

For that purpose the strap 25—referring again to FIG. 7—having been looped around the spring top bar 31 is led down from that bar, around the bar 27, and fed over its own initial run around that bar to emerge as a strap end 24.

The inherent friction between the two runs of the strap 25 as they pass over the bars 27 and 31 has two results. If the end of the strap 25 first fed into the housing (i.e. the lower run in FIG. 7) is fixed, e.g. By spot welding to a clip (not shown) which then plugs into the housing 26 or to another fixed point; and the tail end 24 of the strap is pulled, by contrast, in the direction of the arrow indicated in FIG. 7, then it will slide over its lower run mate; but the friction effect will be such that the top bar 31 of the torsion spring 28 will be pulled down and cause the spring bar 31 to deflect against the energy stored in the spring itself, i.e. against the action of the spring.

A pointer 32 is freely rotatably mounted on a pivot 33 moulded into the bottom housing half 29 of the housing 26. Integrally moulded into the pointer, one on each opposite side of its pivot axis, are thin projections 34, 35 each of which terminates in—and thence joins the pointer to—a respective one of two links 36 and 37. The extremities of each link clip to one of the opposite extending legs 38, 39, which extend from the torsion coils of the spring 28 to join the spring top bar 31.

As the spring bar 31 moves up and down, therefore, the links 36, 37 act via the thin projections 34, 35 to swivel the pointer 32 about its pivot 33. The means by which the extremities of the links 36 and 37 clip to the spring legs 38, 39 can be settled by the skilled reader; similarly the way in which the link extremities can move up and down with the spring legs whilst staying sufficiently relatively in place to exert the necessary swivel action on the pointer 32 will be evident without the need for inventive thought.

In its essential operation this embodiment works the same way as the first embodiment. Pulling the strap end 24 causes the spring bar 31 to move downwards against the action of the torsion twist of the spring 28. Consequent movement of the links 36, 37 causes the connecting projections 34, 35 to swivel the pointer clockwise (when viewed in FIG. 9) and when the pointer end 41 coincides with the reference datum line 42 in the eyes of the user, the required pressure applied by the device has been achieved; the device being of course calibrated in advance for this purpose.

In this embodiment, the way the strap interacts with the spring effectively provides the necessary non-return gate as well as measuring the tensile force in the strap.

1. A device for indicating the pressure applied to an object by a strap attached thereto as the strap, in use, is tensioned; the device being adapted to receive an end of the strap and to allow the strap end to be pulled through the device against the action of a deformable spring means with which, in use, the strap end co-operates so that as the strap end is pulled through the device, thereby progressively increasing the pressure applied to the object, the spring means progressively deflects in proportion to that applied pressure; and with means linking the deflection of the spring means to the movement of a pointer which thus provides an indication of the pressure being applied by the strap.

2. A device according to claim 1 and in which the spring means is resiliently deformable, thereby enabling the device to be reused with the same or another intact strap.

3. A device according to claim 1 and in which the pointer is orientated to move progressively over the width dimension of the strap as the spring deflection increases.

4. A device according to claim 1 and incorporating a non-return gate which, as the strap end is pulled through the device, prevents the strap from loosening its pressure on the object.

5. A device according to claim 4 and in which the non return gate cannot subsequently be released other than by breaking the strap.

6. The combination of a device according to claim 1, and a strap adapted to co-operate therewith, in which a reference datum is marked on that region of the strap which, in use, will pass through the device and be visible by a user when the strap movement ceases.

7. A combination according to claim 6 and in which the datum interacts visually with the pointer to show whether or not a predetermined pressure has been reached or exceeded.

8. A combination according to claim 6 and in which a part of the strap is weakened or otherwise designed to be wholly or partially broken, relatively readily, by a user in order to release the pressure hitherto applied to the object by the strap.

9. A combination according to claim 8 and in which the strap is so designed that breaking it renders the strap unable to be reused.

10. A combination according to claim 6 and in which the strap is made from substantially inextensible material.
